(19) United States
(12) Patent Application Publication
(10) Pub. No.: US 2022/0047753 A1
(43) Pub. Date: Feb. 17, 2022

(54) Air-Disinfecting Photocatalytic Device

(71) Applicant: Aleddra Inc., Renton, WA (US)

(72) Inventors: Chia-Yiu Maa, Bellevue, WA (US); Chun-Te Yu, Bellevue, WA (US)

(21) Appl. No.: 16/995,588

(22) Filed: Aug. 17, 2020

Related U.S. Application Data
(63) Continuation-in-part of application No. 16/991,439, filed on Aug. 12, 2020.

(51) Int. Cl. A61L 9/00

(52) U.S. Cl. CPC A61L 9/00; A61L 2209/12

(57) ABSTRACT

An air-disinfecting photocatalytic device includes a housing, an air-permeable porous carrier with at least two sides, a fan, and a light source. The air-permeable porous carrier contains a photocatalyst material, and the light source activates the photocatalyst material in air-permeable porous carrier. The housing, the air-permeable porous carrier, and the fan together form an air chamber. The fan operates to either increase or deplete the air in the air chamber, resulting an air pressure difference between the air inside and outside the air chamber, and causing the air to pass through the air-permeable porous carrier from the high air pressure side of the air-permeable porous carrier to the low air pressure side of the air-permeable porous carrier. As the air passes through the air-permeable porous carrier, airborne pathogens are trapped on the surface of the air-permeable porous carrier, on which light-activated photocatalyst material kills the pathogens trapped thereon.

### BACKGROUND

The present disclosure is a continuation-in-part (CIP) of U.S. patent application Ser. No. 16/991,439, filed 12 Aug. 2020, the content of which being herein incorporated by reference in its entirety.

### BACKGROUND

### Technical Field

The present disclosure pertains to the field of photocatalytic devices and, more specifically, proposes an air-disinfecting photocatalytic device.

### Description of Related Art

Photocatalysts are known to become active under ultraviolet light and kill bacteria by breaking down the cell wall of the bacteria. Soma, R., et al., in U.S. Pat. No. 6,242,752 teaches the use of a photocatalytic film made of anatase-type titanium dioxide (TiO₂) on the lens of a lighting device such that, as the light originating from the lighting device shines through the titanium oxide film, the UV rays of the light activate the photocatalyst, causing it to break down the bacteria cell wall and resulting in the killing of the bacteria. In U.S. Pat. No. 9,522,384, Liu L. et al. teaches the use of rhombus-shape anatase-type titanium dioxide (TiO₂) such that this new type of TiO₂ can be activated by visible light wavelengths and become germicidal active.

In U.S. Pat. No. 10,118,170, Maa C. et al. (hereinafter “Maa”) teaches an anti-bacterial lighting apparatus where the photocatalytic film is coated on the surface of the lens of a lighting apparatus, and as the light of the light source of the apparatus activates the photocatalytic film on the lens, the photocatalytic film will kill any airborne bacteria or viruses making physical contact with the lens. The limitation with Maa's teaching is that for the airborne pathogens to make physical contact with photocatalytic film, it requires air movement to bring the airborne pathogens to the lens. If there is not sufficient air movement where the lighting apparatus is installed, then the germicidal effect is limited. Moreover, the use of photocatalytic material for disinfecting the air and removing airborne pathogens is not limited to lighting devices.

The present disclosure introduces a new air-disinfecting photocatalytic device that comes with a built-in air circulation mechanism for bringing airborne pathogens through its inside chamber and through an air-permeable porous carrier containing a photocatalytic material such that an internal light source would activate the photocatalyst material for killing the pathogens. The light source may not be used for general lighting. Rather, its primary function is to activate the photocatalyst material. This present disclosure can be used to disinfect airborne pathogens with the benefit of lowering the infection rate of air-transmitted diseases. There is no restriction on the form factor of such device. It may be stationary, portable, or wearable. This device does not generate any ozone.

### SUMMARY

In one aspect, the air-disinfecting photocatalytic device comprises a housing, an air-permeable porous carrier with at least two sides, a fan, and a light source. The housing houses the air-permeable porous carrier, the fan, and the light source. The air-permeable porous carrier contains a photocatalyst material. The light source activates the photocatalyst material in the air-permeable porous carrier. The housing, the air-permeable porous carrier, and the fan together form an air chamber. The fan operates to either increase or deplete the air in the air chamber, resulting in an air pressure difference between a first air pressure inside the air chamber and a second air pressure outside the air chamber. As a result, the air pressure difference causes the air to pass through the air-permeable porous carrier from the high air pressure side of the air-permeable porous carrier to the low air pressure side of the air-permeable porous carrier. The air pressure in the air chamber may be higher or lower, depending on the airflow direction of the fan. As air passing through air-permeable porous carrier, airborne pathogens are trapped on the surface of the air-permeable porous carrier. The photocatalyst material in the air-permeable porous carrier which has been activated by the light source would kill the pathogens trapped on the surface of the air-permeable porous carrier.

In some embodiments, a main active ingredient of the photocatalyst material in the air-permeable porous carrier is titanium dioxide (TiO₂). In some other embodiments, the photocatalyst material contains a secondary active ingredient comprising silver, gold, copper, zinc, nickel, or a combination thereof. These metals when embedded in a photocatalyst are known to enhance the photocatalytic activity with visible light. Alternatively, the metal photocatalyst material may be used without TiO₂ as the main active ingredient of the photocatalyst material. In such cases, titanium dioxide (TiO₂) is not used, and a main active ingredient of the photocatalyst material in the air-permeable porous carrier may be silver, gold, copper, zinc, nickel, or any combination thereof.

In some embodiments, the light source emits light mainly in the 200 nm to 400 nm wavelength range. These are UV light sources which are known to be effective in activating TiO₂ photocatalyst. In some other embodiments, the light source emits light mainly in the 400 nm to 700 nm wavelength range. These light sources could be used in conjunction with TiO₂ mixed a secondary active ingredient comprising silver, gold, copper, zinc, nickel, or a combination thereof.

In some embodiments, the light source may reside in the air chamber, such that the light source is positioned closer to the air-permeable porous carrier and may effectively activate the photocatalyst material in the air-permeable porous carrier. In some other embodiments, light source may reside outside the air chamber such that the light source may be easily replaceable.

In some embodiments, the light source and the air-permeable porous carrier are disposed in a way such that there is no obstruction in a line of sight between the light source and the air-permeable porous carrier. Any obstruction in the line of sight between the light source and the air-permeable porous carrier would reduce the total spectral power received by the air-permeable porous carrier from the light source. In some other embodiments, the lighting angle of the light from the light source to the surface of the air-permeable porous carrier is less than 45 degree. This condition may be necessary as the total spectral power received by the air-permeable porous carrier from the light source would be reduced with a larger lighting angle from the light source to the air-permeable porous carrier.

The effectiveness of the photocatalyst activity of the device depends on the physical contact of the airborne pathogens with the photocatalyst material in the air-permeable porous carrier. When the air-permeable porous carrier is covered with dusts, the photocatalytic killing effectiveness of the device against airborne pathogens will be reduced. Therefore, it is critical for the air-permeable porous carrier to be replaceable, and ideally without any tools. In some embodiments, the air-permeable porous carrier is replaceable by a user without using any tool.

The UV light source tends to have a shorter lifetime, as compared to, for example, the lifetime of the fan. In some embodiments, the light source is replaceable by a user without using any tool. This is so that when the light source expires, it can be easily replaced with a new one, thus extending the lifetime of the device.

In some embodiments, the air-permeable porous carrier comprises non-woven fabric or melt-blown fabric, which is one of the most used air-permeable porous material. The TiO₂ photocatalyst material may be added to non-woven/melt-blown fabric through spraying a TiO₂ solution onto the fabric or through submerging the fabric in a TiO₂ solution. In some other embodiments, the air-permeable porous carrier comprises ceramic. In this case, the TiO₂ photocatalyst material may be added to the ceramic carrier through firstly submerging the carrier in a TiO₂ solution and followed by a heat-curing process. Alternatively, an evaporation process may be used to add TiO₂ coating onto the ceramic carrier.

The most expensive component of the present disclosure is the air-permeable porous carrier. It would accumulate dusts over time as mentioned early, thus reducing the photocatalytic effectiveness against airborne pathogens. Having a design with a replaceable air-permeable porous carrier is thus desirable. What is even better would be a cleanable and reusable air-permeable porous carrier such that it can be taken out of the device for cleaning the dusts on its surface and be reused, saving the cost of getting a new air-permeable porous carrier. The cleaning process may include submerging the carrier into hot water for 15 minutes for reactivating the photocatalyst material.

In another aspect, the air-disinfecting photocatalytic device comprises a housing having an air-permeable portion with at least two sides, a fan, and a light source. The housing is free-standing and requires no additional frame to house the light source. The light source is placed inside the housing. The air-permeable portion of the housing contains a photocatalyst material, and the light source activates the photocatalyst material in the air-permeable portion of the housing. The housing and the fan together form an air chamber. The fan operates to either increase or deplete the air in the air chamber, resulting in an air pressure difference between a first air pressure inside the air chamber and a second air pressure outside the air chamber, thereby causing the air to pass through the air-permeable portion of the housing from the high air pressure side of the air-permeable portion of the housing to the low air pressure side of the air-permeable portion of the housing. The airborne pathogens are trapped on the surface of the air-permeable portion of the housing when air passes through the air-permeable portion of the housing. The photocatalyst material in the air-permeable portion of the housing being activated by the light source kills the pathogens trapped on the surface of the air-permeable portion of the housing.

In some embodiments, a main active ingredient of the photocatalyst material in the air-permeable portion of the housing is titanium dioxide (TiO₂). In some other embodiments, the photocatalytic material may contain a secondary active ingredient comprising silver, gold, copper, zinc, nickel, or a combination thereof. Alternatively, the metal photocatalyst material may be used without TiO₂ as the main active ingredient of the photocatalyst material. In such cases, titanium dioxide (TiO₂) is not used, and a main active ingredient of the photocatalyst material in the air-permeable porous carrier may be silver, gold, copper, zinc, nickel, or any combination thereof.

In some embodiments, the light source emits light mainly in the 200 nm to 400 nm wavelength range. In some other embodiments, the light source emits light mainly in the 400 nm to 700 nm wavelength range.

### BRIEF DESCRIPTION OF THE DRAWINGS

The accompanying drawings are included to aid further understanding of the present disclosure, and are incorporated in and constitute a part of the present disclosure. The drawings illustrate a select number of embodiments of the present disclosure and, together with the detailed description below, serve to explain the principles of the present disclosure. It is appreciable that the drawings are not necessarily to scale, as some components may be shown to be out of proportion to size in actual implementation in order to clearly illustrate the concept of the present disclosure.

FIG. 1 schematically depicts a diagram of an air-disinfection photocatalytic device with a light source inside the air chamber.

FIG. 2 schematically depicts a diagram of an air-disinfection photocatalytic device with a light source outside the air chamber.

FIG. 3 schematically depicts another embodiment of the air-disinfection photocatalytic device with a light source inside the air chamber.

FIG. 4 schematically depicts an embodiment of the air-disinfection photocatalytic device in the form of a traditional lantern.

FIG. 5 schematically depicts an embodiment of the air-disinfection photocatalytic device in the form of a 3D cube.

FIG. 6 schematically depicts an embodiment of the air-disinfection photocatalytic device in the form of a modern lantern design.

### DETAILED DESCRIPTION OF PREFERRED EMBODIMENTS

Overview

Various implementations of the present disclosure and related inventive concepts are described below. It should be acknowledged, however, that the present disclosure is not limited to any particular manner of implementation, and that the various embodiments discussed explicitly herein are primarily for purposes of illustration. For example, the various concepts discussed herein may be suitably implemented in a variety of lighting apparatuses having different form factors.

The present disclosure discloses an air-disinfecting photocatalytic device that has a housing, an air-permeable porous carrier with at least two sides, a fan, and a light source. The air-permeable porous carrier contains a photocatalyst material, and the light source activates the photocatalyst material in air-permeable porous carrier. The housing, the air-permeable porous carrier, and the fan together form an air chamber. The fan operates to either increase or deplete the air in the air chamber, resulting in an air pressure difference between a first air pressure inside the air chamber and a second air pressure outside the air chamber, thereby causing the air to pass through the air-permeable porous carrier from the high air pressure side of the air-permeable porous carrier to the low air pressure side of the air-permeable porous carrier. As the air passes through the air-permeable porous carrier, airborne pathogens are trapped on the surface of the air-permeable porous carrier. On the surface of the air-permeable porous carrier, the photocatalyst material being activated by the light source kills the pathogens trapped on the surface of the air-permeable porous carrier.

Example Implementations

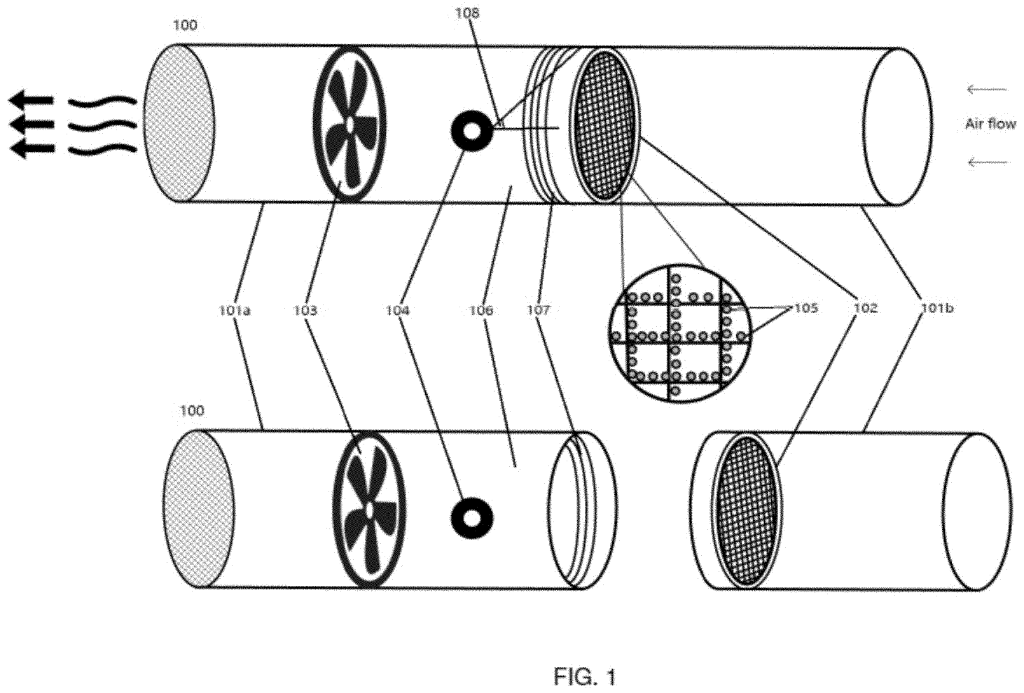

FIG. 1 is an embodiment of the air-disinfecting photocatalytic device of the present disclosure with a cylinder shape 100. The housing 101a, 101b houses the air-permeable porous carrier 102, the fan 103, and the light source 104. The air-permeable porous carrier 102 is made of ceramic and its surface is coated with a photocatalyst TiO₂ 105. When the light source 102 is on, it activates the photocatalyst 105. The housing 101a, 101b, and the air-permeable porous carrier 102, and the fan 103 together form an air chamber 106. The fan 103 operates to draw air away from the air chamber 106 to the left. As a result, the air pressure in the air chamber 106 will drop, forcing the air in the housing section 101b to pass from the right side of the air-permeable porous carrier 102 to the left side of the carrier and into the air chamber. As the air passing through the air-permeable porous carrier 102, the airborne pathogens are trapped on the surface of the carrier, and the photocatalyst TiO₂ 105 being activated by the light source 104 will kill the pathogens trapped on the surface of the carrier.

The light source 104 emits light mainly in the 200 nm to 400 nm wavelength range. When a secondary active photocatalytic ingredient comprising silver, gold, copper, zinc, nickel, or a combination thereof is used in the photocatalyst 105, then the photocatalyst 105 may be activated by visible light. In which case, it is possible to use a visible light source emitting light mainly in the 400 nm to 700 nm wavelength range for the light source 104. In this embodiment the light source 104 is placed inside the air chamber 106. There is no obstruction in a line of sight between the light source 104 and the air-permeable porous carrier 102. Moreover, the lighting angle 108 of the light from the light source 104 to the surface of the air-permeable porous carrier 102 is less than 45 degree, for ensuring sufficient amount of spectral power of the light emitted from the lighting 104 is received by the photocatalyst 105 on the carrier 102.

The two sections of the housing, 101a and 101b, are connected through their threaded segment 107. These two sections of the housing 101a and 10ab can be disengaged by rotating the housing section 101b counterclockwise, without using any tool. Once the housing section 101b is disengaged from the housing section 101a, the air-permeable porous carrier 102 can be replaced with a new carrier or removed for cleaning.

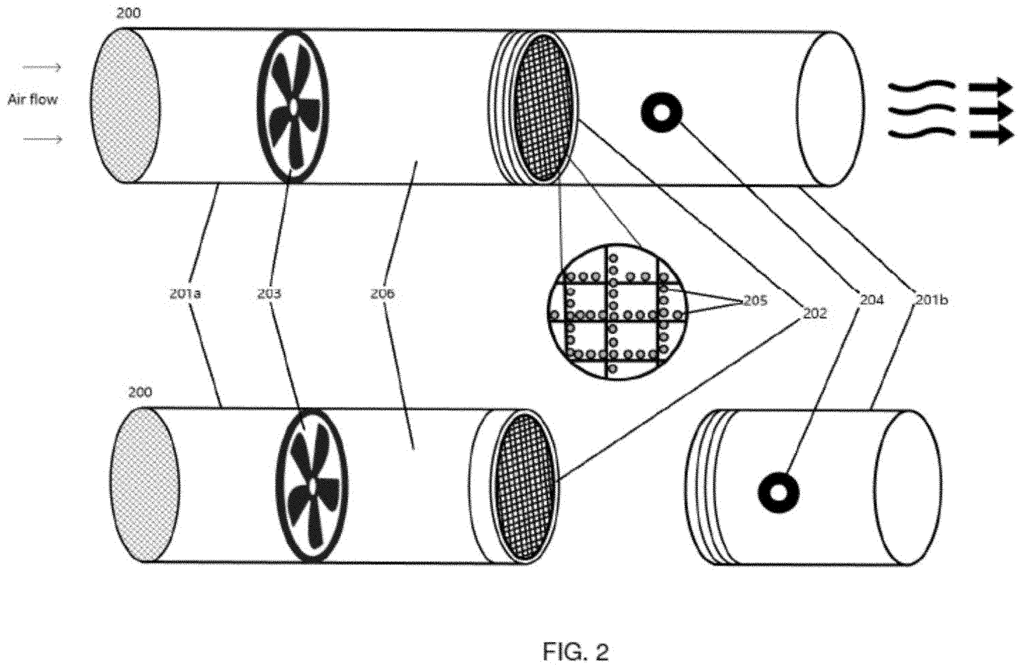

FIG. 2 is another embodiment of the air-disinfecting photocatalytic device of the present disclosure also with a cylinder shape 200. The housing 201a, 201b houses the air-permeable porous carrier 202, the fan 203, and the light source 204. The air-permeable porous carrier 202 is made of ceramic and its surface is coated with a photocatalyst TiO₂ 205. The housing 201a, 201b, and the air-permeable porous carrier 202, and the fan 203 together form an air chamber 206. The fan 203 operates to add air into the air chamber 206. As a result, the air pressure in the air chamber 206 will increase, forcing the air in the air chamber 206 to pass from the left side of the air-permeable porous carrier 202 to the right side of the carrier and into the housing section 201b. As the air passing through the air-permeable porous carrier 202, the airborne pathogens are trapped on the surface of the carrier, and the photocatalyst TiO₂ 205 being activated by the light source 204 will kill the pathogens trapped on the surface of the carrier.

In this embodiment the light source 204 is placed outside the air chamber 206. There is no obstruction in a line of sight between the light source 204 and the air-permeable porous carrier 202. The two sections of the housing, 201a and 201b, are connected through their threaded segment 207. These two sections of the housing 201a and 20ab can be disengaged by rotating the housing section 201b counterclockwise, without using any tool. Once the housing section 201b is disengaged from the housing section 201a, the light source 204 can be replaced with a new one.

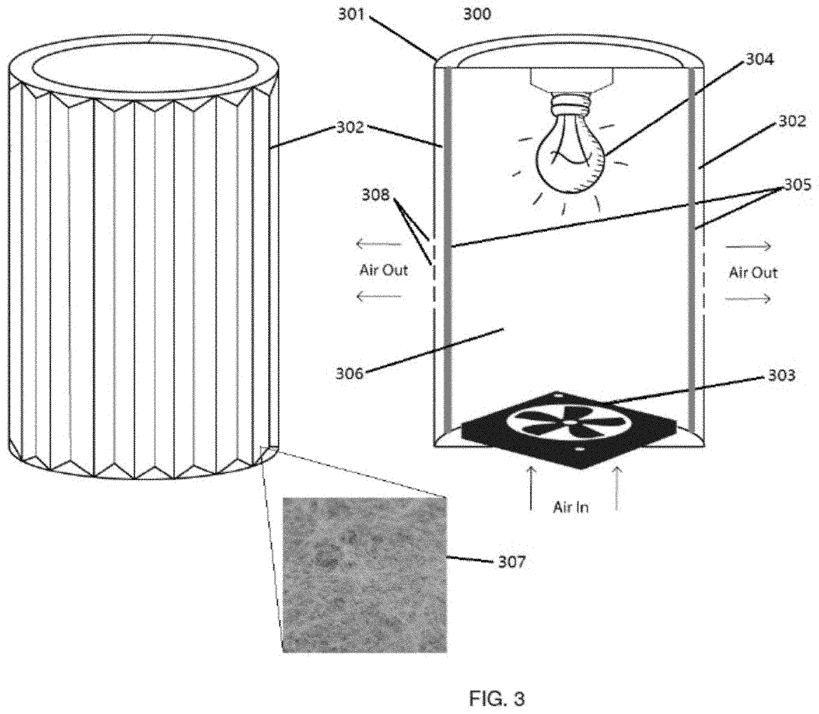

FIG. 3 is another embodiment of the air-disinfecting photocatalytic device of the present disclosure 300. The housing 301 houses the air-permeable porous carrier 302, the fan 303, and the light source 304. The air-permeable porous carrier 302 is made of melt-blown fabric 307 and its surface is coated with a photocatalyst TiO₂ 305. The housing 301, and the air-permeable porous carrier 302, and the fan 303 together form an air chamber 306. The fan 303 operates to add air into the air chamber 306. As a result, the air pressure in the air chamber 306 will increase, forcing the air in the air chamber 306 to pass inner side of the air-permeable porous carrier 302 to the outer side of the carrier and then through the holes 308 on the housing 301. As the air passing through the air-permeable porous carrier 302, the airborne pathogens are trapped on the surface of the carrier, and the photocatalyst TiO₂ 305 being activated by the light source 304 will kill the pathogens trapped on the surface of the carrier.

In this embodiment the light source 304 is placed inside the air chamber 306. There is no obstruction in a line of sight between the light source 304 and the air-permeable porous carrier 302. The housing 301 may be lifted from the top without using any tool so that the air-permeable porous carrier 302 and the light source 304 may be replaced.

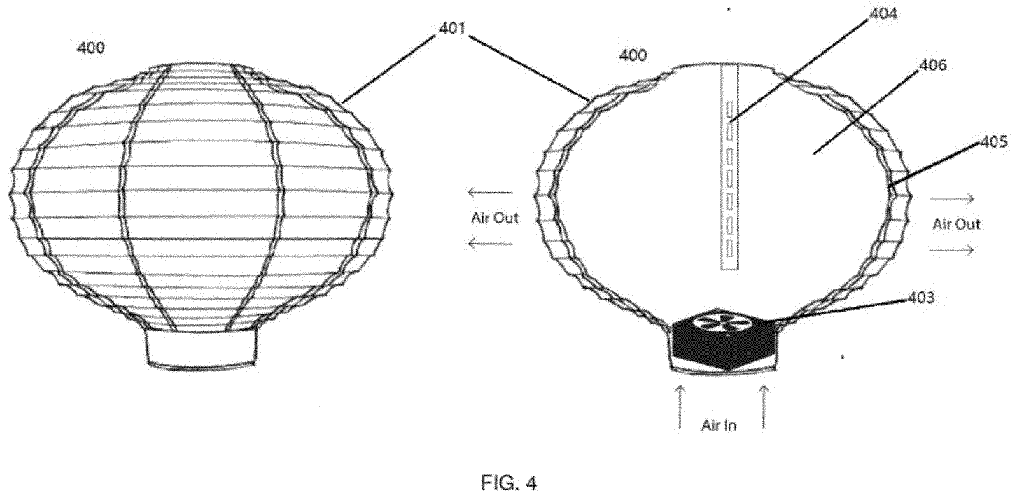

FIG. 4 is another embodiment of the air-disinfecting photocatalytic device of the present disclosure 400 in the form of a traditional lantern. The device has a housing 401, a fan 403, and a light source 404. The housing 401 houses has an air-permeable portion with at least two sides: the inner side and the outer side. The housing 401 is free-standing and requires no additional frame to house the light source 404. The light source 404 is placed inside the air-permeable portion of the housing 401. The air-permeable portion of the housing contains a photocatalyst TiO₂ 405. The light source 404 activates the photocatalyst material in the air-permeable portion of the housing 401. The housing 401 and the fan 403 together form an air chamber 406. The fan 403 operates to add air into the air chamber 406. As a result, the air pressure in the air chamber 406 will increase, forcing the air in the air chamber 406 to pass inner side of the air-permeable portion of the housing 401 to the outer side of the housing. As the air passing through the air-permeable portion of the housing 401, the airborne pathogens are trapped on the surface of the air-permeable portion of the housing 401, and the photocatalyst TiO₂ 405 being activated by the light source 404 will kill the pathogens trapped on the surface of the air-permeable portion of the housing 401.

The light source 404 emits light mainly in the 200 nm to 400 nm wavelength range. When a secondary active photocatalytic ingredient comprising silver, gold, copper, zinc, nickel, or a combination thereof is used in the photocatalyst 405, then the photocatalyst 405 may be activated by visible light. In which case, it is possible to use a visible light source emitting light mainly in the 400 nm to 700 nm wavelength range for the light source 404.

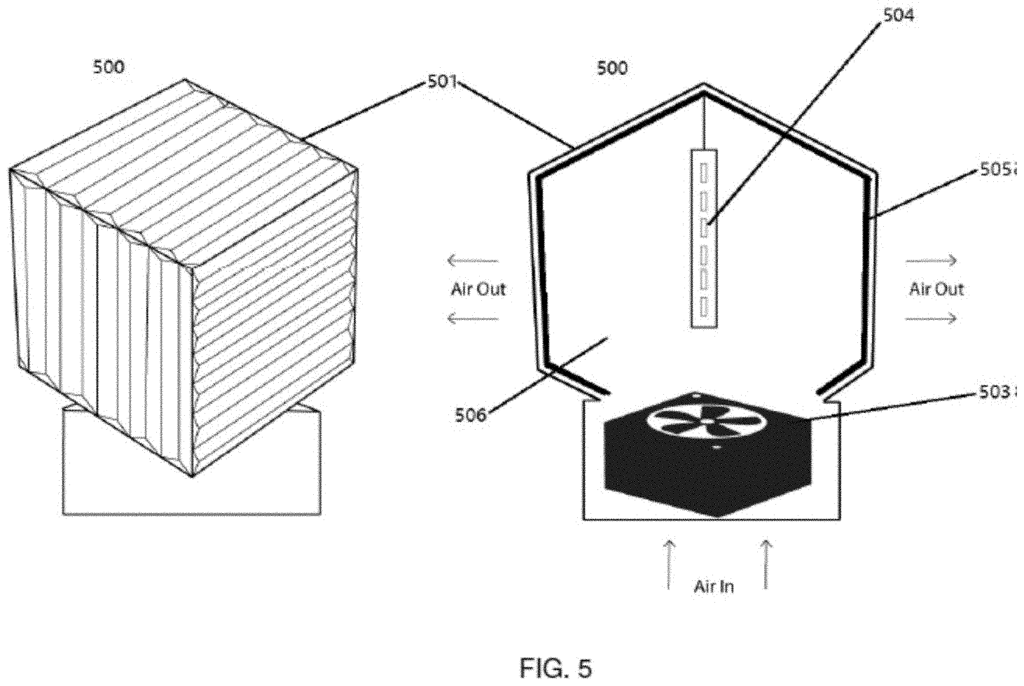

FIG. 5 is another embodiment of the air-disinfecting photocatalytic device of the present disclosure 500 in the form of a 3D cube. The device has a housing 501, a fan 503, and a light source 504. The housing 501 houses has an air-permeable portion with at least two sides: the inner side and the outer side. The housing 501 is free-standing and requires no additional frame to house the light source 504. The light source 504 is placed inside the air-permeable portion of the housing 501. The air-permeable portion of the housing contains a photocatalyst TiO₂ 505. This embodiment 500 operates in a similar fashion as the embodiment 400.

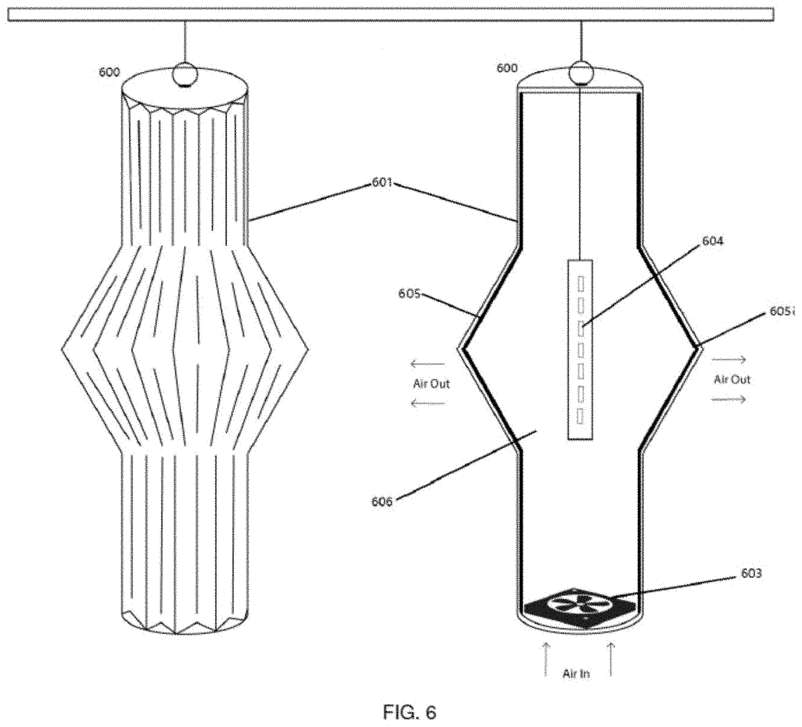

FIG. 6 is another embodiment of the air-disinfecting photocatalytic device of the present disclosure 600 in the form of a modern lantern. The device has a housing 601, a fan 603, and a light source 604. The housing 601 houses has an air-permeable portion with at least two sides: the inner side and the outer side. The housing 601 is free-standing and requires no additional frame to house the light source 604. The light source 604 is placed inside the air-permeable portion of the housing 601. The air-permeable portion of the housing contains a photocatalyst TiO₂ 605. This embodiment 600 operates in a similar fashion as the embodiment 400.

Additional and Alternative Implementation Notes

Although the techniques have been described in language specific to certain applications, it is to be understood that the appended claims are not necessarily limited to the specific features or applications described herein. Rather, the specific features and examples are disclosed as non-limiting exemplary forms of implementing such techniques.

As used in this application, the term “or” is intended to mean an inclusive “or” rather than an exclusive “or.” That is, unless specified otherwise or clear from context, “X employs A or B” is intended to mean any of the natural inclusive permutations. That is, if X employs A; X employs B; or X employs both A and B, then “X employs A or B” is satisfied under any of the foregoing instances. In addition, the articles “a” and “an” as used in this application and the appended claims should generally be construed to mean “one or more,” unless specified otherwise or clear from context to be directed to a singular form.

What is claimed is:

1. An air-disinfecting photocatalytic device, comprising
a housing;
an air-permeable porous carrier with at least two sides;
a fan;
a light source,
wherein:
the housing houses the air-permeable porous carrier, the fan, and the light source,
the air-permeable porous carrier contains a photocatalyst material,
the light source emits a light to activate the photocatalyst material in the air-permeable porous carrier,
the housing, the air-permeable porous carrier, and the fan together form an air chamber,
the fan operates to either increase or deplete an amount of air in the air chamber, resulting in an air pressure difference between a first air pressure inside the air chamber and a second air pressure outside the air chamber, thereby causing air to pass through the air-permeable porous carrier from a high air pressure side of the air-permeable porous carrier to a low air pressure side of the air-permeable porous carrier,
airborne pathogens are trapped on a surface of the air-permeable porous carrier when the air passes through the air-permeable porous carrier, and
the photocatalyst material in the air-permeable porous carrier being activated by the light source kills the pathogens trapped on the surface of the air-permeable porous carrier.

2. The air-disinfecting photocatalytic device of claim 1, wherein a main active ingredient of the photocatalyst material in the air-permeable porous carrier is titanium dioxide (TiO₂).

3. The air-disinfecting photocatalytic device of claim 2, wherein the photocatalyst material contains a secondary active ingredient comprising silver, gold, copper, zinc, nickel, or a combination thereof.

4. The air-disinfecting photocatalytic device of claim 1, wherein the light source emits the light with a wavelength in a range of 200 nm to 400 nm.

5. The air-disinfecting photocatalytic device of claim 1, wherein the light source emits the light with a wavelength in a range of 400 nm to 700 nm.

6. The air-disinfecting photocatalytic device of claim 1, wherein the light source resides in the air chamber.

7. The air-disinfecting photocatalytic device of claim 1, wherein the light source resides outside the air chamber.

8. The air-disinfecting photocatalytic device of claim 1, wherein the light source and the air-permeable porous carrier are disposed in a way such that there is no obstruction in a line of sight between the light source and the air-permeable porous carrier.

9. The air-disinfecting photocatalytic device of claim 1, wherein a lighting angle of the light from the light source to the surface of the air-permeable porous carrier is less than 45 degree.

10. The air-disinfecting photocatalytic device of claim 1, wherein the air-permeable porous carrier is replaceable without using any tool.

11. The air-disinfecting photocatalytic device of claim 1, wherein the light source is replaceable without using any tool.

12. The air-disinfecting photocatalytic device of claim 1, wherein the air-permeable porous carrier comprises non-woven fabric or melt-blown fabric.

13. The air-disinfecting photocatalytic device of claim 1, wherein the air-permeable porous carrier comprises ceramic.

14. The air-disinfecting photocatalytic device of claim 1, wherein the air-permeable porous carrier is cleanable and reusable.

15. An air-disinfecting photocatalytic device, comprising
a housing having an air-permeable portion with at least two sides;
a fan;
a light source,
wherein:
the housing is free-standing and requires no additional frame to house the light source,
the light source is placed inside the air-permeable portion of the housing that contains a photocatalyst material,
the light source emits a light to activate the photocatalyst material in the air-permeable portion of the housing,
the housing and the fan together form an air chamber,
the fan operates to either increase or deplete an amount of air in the air chamber, resulting in an air pressure difference between a first air pressure inside the air chamber and a second air pressure outside the air chamber, thereby causing air to pass through the air-permeable portion of the housing from a high air pressure side of the air-permeable portion of the housing to a low air pressure side of the air-permeable portion of the housing,
airborne pathogens are trapped on a surface of the air-permeable portion of the housing when the air passes through the air-permeable housing, and
the photocatalyst material in the air-permeable portion of the housing being activated by the light source kills the pathogens trapped on the surface of the air-permeable portion of the housing.

16. The air-disinfecting photocatalytic device of claim 15, wherein a main active ingredient of the photocatalyst material in the air-permeable portion of the housing is titanium dioxide (TiO₂).

17. The air-disinfecting photocatalytic device of claim 16, wherein the photocatalytic material contains a secondary active ingredient comprising silver, gold, copper, zinc, nickel, or a combination thereof.

18. The air-disinfecting photocatalytic device of claim 15, wherein the light source emits the light with a wavelength in a range of 200 nm to 400 nm.

19. The air-disinfecting photocatalytic device of claim 15, wherein the light source emits the light with a wavelength in a range of 400 nm to 700 nm.

20. The air-disinfecting photocatalytic device of claim 15, wherein a main active ingredient of the photocatalyst material in the air-permeable porous carrier comprises silver, gold, copper, zinc, nickel, or a combination thereof.

21. The air-disinfecting photocatalytic device of claim 1, wherein a main active ingredient of the photocatalyst material in the air-permeable porous carrier comprises silver, gold, copper, zinc, nickel, or a combination thereof.
